(19) United States
(12) Patent Application Publication
(10) Pub. No.: US 2011/0224745 A1
(43) Pub. Date: Sep. 15, 2011

(54) Emergency Medical Station And Advertisement Display

(76) Inventor: David C. Magruder, Tequesta, FL (US)

(21) Appl. No.: 12/719,964

(22) Filed: Mar. 9, 2010

(51) Int. Cl. A61N 1/39; G09B 23/28

(52) U.S. Cl. 607/5; 434/265

(57) ABSTRACT

A medical emergency station having a housing, an automated external defibrillator in the housing, and a video screen incorporated into a single unit. The medical emergency station includes a video regarding the proper use of the automated external defibrillator. The video is activated whenever the automated external defibrillator is removed from the housing. A commercial advertisement and other media appears on the video screen whenever the video regarding the use of the automated external defibrillator is not in use. The commercial advertisements help to offset the cost of the medical emergency station.

### FIELD OF THE INVENTION

The present invention relates to medical devices, and in particular a system for use in the treatment of an individual who has suffered a cardiac arrest.

### BACKGROUND OF THE INVENTION

Businesses, arenas, concert venues, sports venues, gyms, and other places where a large number of the public attend are concerned with the treatment of individuals who may suffer from a medical emergency. In certain cases, such as sudden cardiac arrest (SCA), it is imperative that treatment begin immediately in order to prevent the individual from dying, and to prevent further damage to the heart. The ability of trained medical personnel such as paramedics to quickly reach an individual who has suffered a heart attack in large areas such as concert venues, sports arenas and large box stores is almost nil.

The most effective treatment for an individual who has suffered a sudden cardiac arrest or arrhythmia is the use of a defibrillator to restore the heart to its normal rhythm and contractive function. There are several classes of defibrillators, including manual defibrillators, implantable defibrillators, semi-automatic defibrillators and automatic defibrillators. The semi-automatic and automatic defibrillators measure an electrocardiogram of the individual to determine if a defibrillation shock is required. The semi-automatic version will prompt an individual to apply the shock while the automatic version will apply the shock as soon as it determines that one is required.

Recently these semi-automatic and automatic defibrillators have become available to the general public. The use of these defibrillators requires some training, and many arenas, sports venues and large stores train their employees in the use of these devices. Many individuals forget how to use these defibrillators if they are not trained on a regular basis or may not use the defibrillators. In addition, these semi-automatic and automatic defibrillators can be properly used by almost anyone with a little assistance or guidance during their use. Some companies have prepared training videos for their employees in the proper use of these semi-automatic and automatic defibrillators. These videos can be shown whenever it is necessary to train someone, and do not require the presence of an individual trained in the use of these defibrillators.

What is needed in the art is a readily accessible automatic defibrillator which can be easily placed in venues such as concerts, sports arenas, gyms, small medical offices, waiting rooms, small fast-food restaurants, hospitality venues, etc.

### DESCRIPTION OF THE PRIOR ART

U.S. Published Application No. 2005/0246199 discloses a health and wellness station which includes a plurality of different emergency response resources incorporated into a single station. An automated external defibrillator, a bioterror response kit, a first aid kit, a fire extinguisher, a security system and an oxygen dispenser are provided in the station. The components of the station are monitored for maintenance and an indicator is activated when maintenance is required.

U.S. Published Application No. 2009/0204161 discloses an automatic defibrillator which measures the ECG of a patient to determine if an electrical shock is required. If the shock is required, the user is prompted to press a button. In the event the button is not pressed, the device, after a predetermined time, will deliver the electrical shock to the patient.

What is needed in the art is an emergency medical station which includes an automated external defibrillator and an instructional video on the proper use of the automated external defibrillator. These emergency medical stations should be self contained so that they can be readily located in areas with public access, such as retail stores, sports arenas, hotels, places of business, etc.

### SUMMARY OF THE INVENTION

A medical emergency station having a housing, an automated external defibrillator in the housing and a video screen are incorporated into a single unit. The medical emergency station includes a video regarding the proper use of the automated external defibrillator. The video is activated whenever the automated external defibrillator is removed from the housing. A commercial advertisement and other media appear on the video screen whenever the video regarding the use of the automated external defibrillator is not in use. The commercial advertisements help to offset the cost of the medical emergency station. Whenever the instructional video is not displayed on the screen other media can be displayed. For example, commercial and public service advertisements and messages, venue schedules, maps and directions to various destinations, weather, financial information, such as stock prices, news alerts, emergency messaging, Amber and Silver alerts, health wellness and beauty information, announcements, sports scores, corporate training and communication, live broadcast feeds, affinity marketing, menus, and/or medical information. These media and commercials help to offset part or all of the cost of placing the medical emergency station at a particular location.

Accordingly, it is an objective of the instant invention to provide an emergency medical station including an automated external defibrillator.

It is a further objective of the instant invention to provide an emergency medical station with an automated external defibrillator with an instructional video regarding the proper use of the automated external defibrillator.

It is yet another objective of the instant invention to provide commercial advertisements on a video screen of a medical emergency station to offset the costs of the emergency medical station.

It is a still further objective of the invention to provide an emergency program at the location of a medical emergency station which includes personnel that are trained in the proper use of automated external defibrillators.

It is a till further objective of the invention to provide a comprehensive automatic external defibrillator (AED) management program inclusive of employee training in the use of automatic external defibrillators.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a front perspective view of the present invention with an advertisement on a video screen;

FIG. 2 is a front view of the present invention illustrating an automated external defibrillator in a housing;

FIG. 3 is a front perspective view of the present invention with an individual using the automated external defibrillator; and

FIG. 4 is an alternative embodiment of the present invention.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

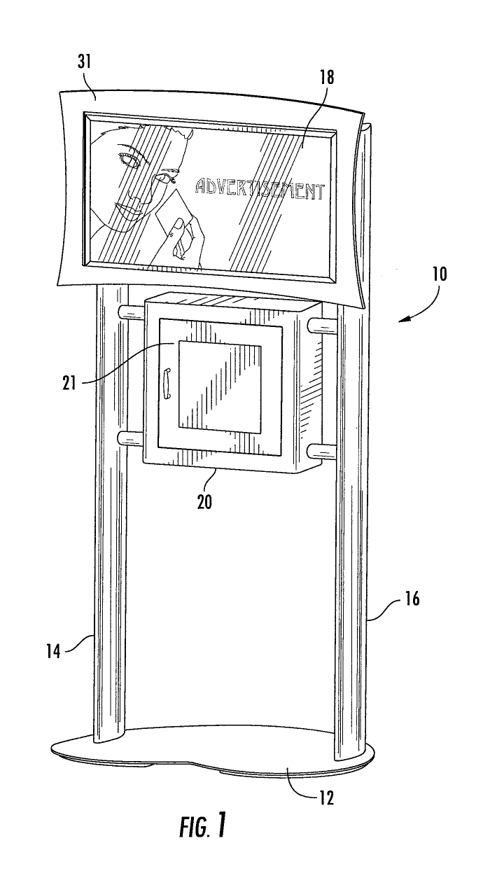

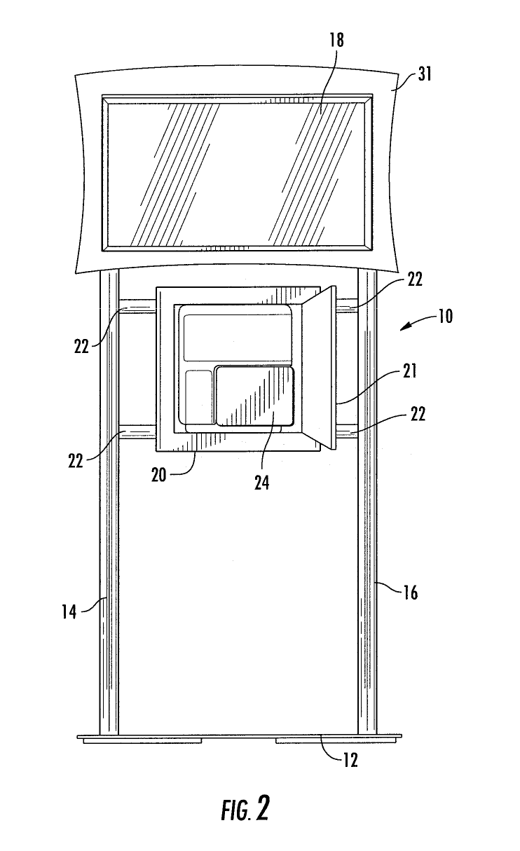

Referring now to FIGS. 1-4, a medical emergency station and method of its use will now be described. Recently automated external defibrillators (AED) have been developed which enable persons with limited medical experience or skill to use them. Many large corporations, health clubs, universities, etc. have installed automated external defibrillators. While the presence of an AED will generally result in saving someone who has suffered a heart attack or cardiac arrest, many individuals are reluctant to use them because they have not received proper training or have forgotten their training.

It has been determined that after 4-6 minutes from when an individual has suffered cardiac arrest the odds of resuscitating the individual or providing other favorable outcomes is greatly diminished. The chances of survival are reduced 7-10% for each minute of delay. The time delay in having emergency medical personal with the proper equipment reaching the victim can be critical.

The present invention will make automated external defibrillators readily available to members of the public along with instructions regarding the proper use of these defibrillators. This should enable the ordinary person to correctly treat an individual who has suffered a heart attack or a cardiac arrest and avoid the time delay waiting for medical specialists with the proper equipment to arrive and provide the proper treatment. The present invention will enable defibrillators and instructions regarding their use to be placed in various locations such as retail stores, shopping malls, sports arenas, concert arenas, health clubs, business complexes, industrial facilities, factories, schools, universities, airports, bus and train depots, rest stops along highways, amusement parks, resorts, etc.

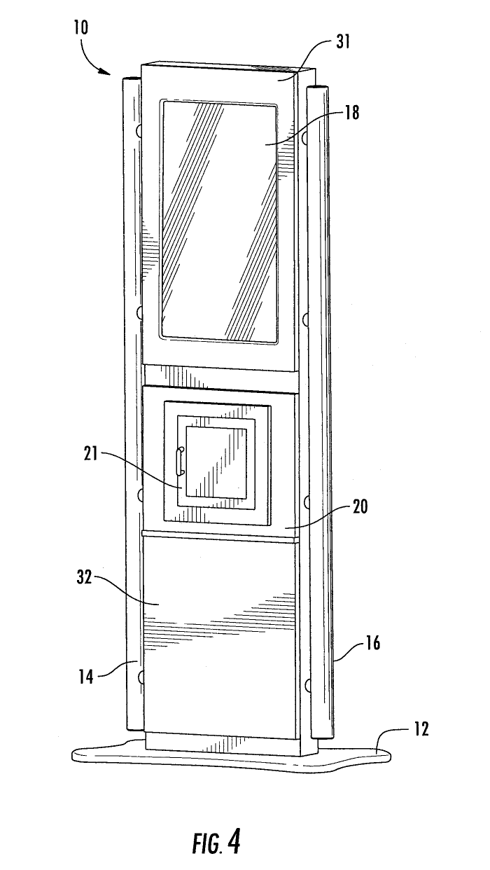

The present invention includes a stand alone emergency medical station 10. The emergency medical station 10 includes a base 12. A plurality of upright supports 14, 16 are secured to the base 12. While two upright supports are illustrated in a preferred embodiment, any number of upright supports, including one, can be employed. The base may include fastening devices, not shown, which enable the base to be secured to an underling surface. This securement can prevent the emergency medical station from tipping over if it is pushed or struck by a large force, such as wind. A visual display, preferably in the form of a video screen 18, is secured between the upright supports 14, 16 and at a top portion of the supports. The height of the visual display above the ground is variable. While the video screen is illustrated as rectangular and horizontally positioned, it could also be rectangular and vertically positioned, square, round, oval or any other shape. FIG. 4 illustrates an alternative embodiment of the present invention wherein the video screen 18 is vertically positioned. The area 32 below the housing 20 can be used to store items or it can be left vacant for appearance purposes only.

A housing 20 is secured between upright supports 14 and 16. The housing has a door 21 which permits entry into the housing and conceals the contents of the housing when they are not in use. The housing is preferably secured to the upright supports 14, 16 by members 22. Four members 22 are illustrated in a preferred embodiment. However, any number of members 22 can be utilized to secure housing 20 to the upright supports 14, 16. While the housing 20 is illustrated as positioned below the video screen, it could also be located above the screen or on either side of the screen. A defibrillator 24 is contained within the housing 20. The defibrillator 24 is preferably an automated external defibrillator. These devices are very portable and once they are removed from the housing 20, they can readily be carried to an individual 28 who has suffered a sudden cardiac arrest. The defibrillator includes indicators, not shown, which indicate the state of readiness of the defibrillator and the different functions that the defibrillator can perform. For example, if the power supply or battery of the defibrillator does not have sufficient electrical power to operate the defibrillator, an indicator, such as a light, will be activated to alert an individual that the defibrillator will not perform its function. Also automated external defibrillators are capable of analyzing an individual's ECG heart rhythm to determine if an individual's arrhythmia is treatable with electrical defibrillation. If the automated external defibrillator determines that the arrhythmia is treatable, an indicator light or sound will be activated so that the operator of the defibrillator can begin. Electrodes 26 are electrically connected to the defibrillator 24. These electrodes are placed on an individual 28 who has a suffered sudden cardiac arrest. These electrodes are capable of measuring an individual's ECG heart rhythm to determine their arrhythmia.

Once a determination is made by the defibrillator that an individual 28 will benefit from electrical defibrillation, a signal is activated so that the operator 30 of the defibrillator will now activate the defibrillator and provide an electrical defibrillation to the individual. A common indicator employed to notify the operator of the defibrillator is a light with the message “shock now”. Other types of indicators can also be employed.

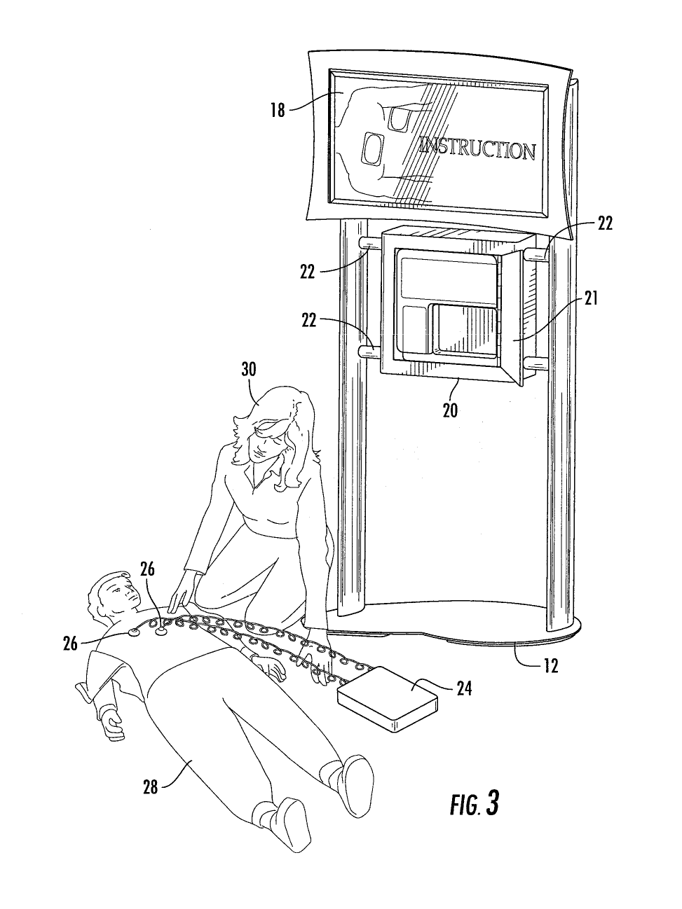

Whenever the housing door 21 is opened, an instructional video is displayed on screen 18. This video is designed to guide a novice user of a defibrillator through all of the steps required to properly use an automatic external defibrillator. The instructional video may be a continuous video or preferably it is designed with built in pauses which permits the operator of the defibrillator to proceed with the video only after a certain procedure is completed. For example, at first the electrodes 26 must be properly placed on the individual 28 so that the individual's ECG heart rhythm can be measured. The video will illustrate the proper location of the electrodes on the individual, as shown in FIG. 3, and then pause until the operator indicates that he/she has accomplished the proper placement of the paddles. The video will then inform the operator that they should apply the electrical defibrillation when the defibrillator indicates it proper to do so. The electrical defibrillation may be applied more than once. Further, employee training on the proper use of automatic external defibrillators can also be implemented in work places and offices. This video quickly demonstrates that there are no more than four basic steps required to properly use an automatic external defibrillator. The video is relatively short, usually less than 30 seconds. It contains the basic requirements for someone to properly administer an automatic external defibrillator to a victim who has suffered a sudden cardiac arrest. The video reminds the individual to call 911, how to determine if a person is lifeless and how to turn on the automatic external defibrillator. Once the automatic external defibrillator has been turned on, it will instruct the user regarding the remaining steps to the AED's proper use. Whenever the instructional video is not displayed on the screen, other media can be displayed, for example; commercial and public service advertisements and messages, venue schedules, maps and directions to various destinations, weather, financial information, such as stock prices, news alerts, emergency messaging, Amber and Silver alerts, health wellness and beauty information, announcements, sports scores, corporate training and communication, live broadcast feeds, affinity marketing, menus, and/or medical information. These media and messages can be displayed or activated as pre-programmed static media, streaming media, touch screen interaction, wireless and via smart card and other smart technologies. These media and commercials help to offset part or all of the cost of placing the emergency medical station at a particular location.

Opening the door 21 can also activate a call to the alarm company which monitors the building in which the emergency medical station is located. The alarm company, upon receiving notification that the door 21 of the housing has been opened, can then call the local Emergency Medical Service provider. In addition, the instructional video will remind the operator 30 that someone should call “911” or an Emergency Medical Service provider operator before proceeding with any treatment. The instructional video can also indicate that cardiac pulmonary resuscitation (CPR) may be employed subsequent to defibrillation and after the paddles are removed from the individual. In addition, if the emergency medical station is located at a place of business, employee education program can be developed and implemented to train the employees on the proper use of the emergency medical station and AED.

The cost of these emergency medical stations may prohibit their use and placement in numerous locations. To offset the cost of these stations, commercial advertisements can be displayed on the video screen. The sale of advertisements will pay for the initial cost and maintenance of the medical stations 10. Whenever the stations 10 are placed in public areas such as sports arenas, shopping malls, airports, etc. various different advertisements can be displayed on the video screen. Whenever the stations 10 are placed in retail stores, sports arenas, health clubs, resorts, etc., advertisements pertaining to that particular location may be displayed on the screen. For example, when the station is placed in a retail store, items that are on sale or that the store is currently promoting can be displayed on the video screen 18. When the station 10 is placed in a health club, the different programs available at the health club and different products available for sale at the health club can be displayed on the video screen 18. When the stations 10 are placed in transportation areas, such as airports, the arrival and departure times can be displayed on the video screen in addition to commercial advertisements.

As illustrated in FIGS. 1-4 the emergency medical station 10 is portable and can be moved from one location to another. The emergency medical station can also be permanently mounted in a particular location. Further, in addition to being mounted on the upright supports 14, 16, the video screen 18 and housing 20 can be mounted onto a wall. The components of the station 10 can be made weather-proof so that the station 10 can also be placed outdoors. This would be advantageous in areas where most of the activity occurs out of doors. For example, these emergency medical stations can be strategically located in certain areas on ski slops, race tracks, swimming pools, tennis courts, etc.

In addition to the advertisement on the video screen, a company logo or store logo/trademark 31 can be displayed surrounding the screen 18. Also, if the station is located at a school or university, the school's logo or mascot could be displayed at 31 surrounding the video screen 18. In addition to a free standing AED and video screen, the unit can be mounted on a wall or reduced in size so as to be portable, thus enabling its ready transportation from one venue to another or from one area at a particular venue to another area.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method of deploying a cardiac arrest emergency device comprising:
providing an automated external defibrillator;
providing a station, said station containing said automated external defibrillator and a video screen;
providing a housing which contains said automated external defibrillator, said housing being secured to said station;
providing an instructional video on the use of said automated external defibrillator, said instructional video appearing on said video screen; and
activating said instructional video upon opening of said housing and activation of said automated external defibrillator.

2. The method of claim 1 wherein said instructional video includes all of the steps to properly use said automated external defibrillator on an individual who has suffered cardiac arrest.

3. The method of claim 1 including presenting a commercial advertisement or other information on said video screen when said automated external defibrillator is not in use.

4. The method of claim 1 including placing said station in an area having access to the public.

5. The method of claim 1 wherein said automated external defibrillator includes a program which determines if an individual has suffered a cardiac arrest;
upon the determination that an individual has suffered a cardiac arrest, said program determining the amount and duration of the electrical energy to be applied to an individual and subsequently applying said proper amount of electrical energy.

6. The method of claim 1 including automatically calling emergency medical specialists when said automated external defibrillator is in use.

7. The method of claim 6 wherein said station includes a door; notifying said emergency medical specialists upon opening of said door.

8. The method of claim 7 including notifying an alarm system in a venue upon opening of said door.

9. The method of claim 6 including developing an emergency program including selection of personnel to assist an individual using said automated external defibrillator and serve as liaisons with said emergency medical specialists.

10. The method of claim 9 including providing training to said personnel on the proper use of said automated external defibrillator.

11. The method of claim 9 including implementing an automatic external defibrillator employee training program.

12. A medical emergency and advertisement station comprising:
a base;
a plurality of upright supports secured to said base;
a housing secured to said upright supports;
a visual display secured to said upright supports;
an audible output device secured to said upright supports; and
a defibrillator secured within said housing;
whereby when said defibrillator is activated a set of instructions on the use of said defibrillator appears on said visual display and on said audio output.

13. The medical emergency and advertisement station of claim 12 including a commercial advertisement being displayed on said visual display when said set of instructions on the use of said defibrillator are not being displayed.

14. The medical emergency and advertisement station of claim 12 including a housing containing said defibrillator, said housing being positioned between said upright supports.

15. The medical emergency and advertisement station of claim 14 wherein said visual display is positioned above said housing.
